(19) United States
(12) Patent Application Publication
(10) Pub. No.: US 2004/0158285 A1
(43) Pub. Date: Aug. 12, 2004

(54) Pressure sock

(76) Inventor: Jayamdiran Pillai, Helderkruin (ZA)

Correspondence Address: FOLEY AND LARDNER, SUITE 500, 3000 K STREET NW, WASHINGTON, DC, 20007, US

(21) Appl. No.: 10/475,134

(22) PCT Filed: Apr. 18, 2002
(86) PCT No.: PCT/ZA02/00067

(30) Foreign Application Priority Data
Apr. 19, 2001 (ZA) 2001/2317

(51) Int. Cl. A61B 17/00

(52) U.S. Cl. 606/204

(57) ABSTRACT

This invention relates to a pressure sock and more particularly, but not exclusively, to a pressure sock for use in improving blood circulation in the lower legs of passengers during aeroplane flights. The pressure sock includes a inflatable bladder attachable to a least part of a bodily extremity such as a lower leg or sole of the foot of a passenger. The pressure sock also includes a manually operable air pump for inflating the bladder to apply pressure to the part of the bodily extremity with which the bladder is in contact.

### FIELD OF THE INVENTION

This invention relates to a pressure sock arrangement and more particularly, but not exclusively, to a pressure sock arrangement for use in improving blood circulation in the lower legs of passengers during aeroplane flights.

### BACKGROUND TO THE INVENTION

Extremity pumps used with inflatable sleeves that fit around a bodily extremity are well known in the field for combating a variety of edema conditions. These pumps and sleeves have multiple chambers for creating a pressure gradient from the distal to proximal end of the bodily extremity. They further have automated pumps with adjustable pressure control and inflation/deflation cycles making them expensive and complicated to use.

Graded pressure or compression socks are also used to help prevent deep vein thrombosis (“DVT”) or so-called “economy class syndrome”. These socks are however not inflatable and deflatable and exert constant pressure on the feet, ankles and calves.

### OBJECT OF THE INVENTION

It is an object of the present invention to provide a pressure sock of the type described above.

### SUMMARY OF THE INVENTION

In accordance with this invention there is provided a pressure sock arrangement comprising two inflatable bladders each attachable to a foot of the human body and a manually operable air pump in fluid flow communication with the bladders for inflating the bladders to apply pressure to the feet to which the bladders are attached, the bladders also being in fluid flow communication.

There is provided for the pressure sock arrangement to include attachment means for attaching the bladders to the feet.

A further feature of the invention provides for the attachment means to be straps having a releasably securable fasteners.

There is further provided for the fasteners to be complementary hook and pile fasteners.

A yet further feature of the invention provides for the bladders to be attached to an operatively upper surface of a sole portions of the pressure sock. The sole portions can be rigid or flexible or the bladder walls can alternatively be dimensioned and shaped to form a sole portion.

A still further feature of the invention provide for the bladder to include a pressure release valve so that an inner cavity of each bladder is in flow communication through the pressure release valve with the outside.

Alternatively, the pressure release valve can be replaced with a slow release valve or each bladder can, in addition to the pressure release valve, include a slow release valve.

In an alternative embodiment each bladder is dimensioned to fit over at least part of a foot.

A yet further feature of the invention provide for each bladder to include protrusions on at least part of its operatively inner surface which contacts part of the bodily extremity when in use.

There is also provided for a pad to be located in sole of the bladder.

The inner cavity of the bladder may be divided into a number of compartments.

A further feature of the invention provides for the compartments to be in flow communication through a number of pressure release valve so that the bladder can be inflated to maintain different pressures in different compartments.

At least one of the compartments may be in flow communication through a pressure release valve or slow release valve with the ambient air outside the pressure socks.

The internal compartments are adapted to maintain a negative pressure gradient from a distal portion to a proximal portion of the bodily extremity.

A still further aspect of the invention provides for the pressure sock arrangement to include a pads, located on the distal portion of the bladders, onto which pressure can be exerted to stimulate airflow between internal compartments within each bladder.

In one embodiment of the invention the pad is comprised of a resiliently deformable material such as sponge or rubber. Alternatively, the pad is an air-filled compartment onto which pressure can be exerted to stimulate air flow from the air filled compartment to the main bladder and back to the air filled compartment in case of negative pressure thus causing pressure change within the main bladder.

These and other features of the invention are described in more detail below.

### BRIEF DESCRIPTION OF THE DRAWINGS

Preferred embodiments of the invention are described below, by way of example only, and with reference to the accompanying drawings in which:

FIG. 1 is a schematic side view of a first embodiment of a pressure sock according to the invention;

FIG. 2 is a schematic side view of a second embodiment of a pressure sock according to the invention; and

FIG. 3 is a perspective view of a third embodiment of a pressure sock in accordance with the invention.

### DETAILED DESCRIPTION OF THE DRAWINGS

With reference to the drawings, in which like features are indicated by like numerals, a pressure sock is generally indicated by reference numeral 10.

The pressure sock 10 consists of an inflatable bladder 12 dimensioned to fit a foot 15, ankle 17 and part of a calf 19; a manually operable air pump 14 for inflating the bladder 12; a flow passage which extends from inside the air pump 14 through a tube 16 and terminates in an inner cavity or inner compartment 13 of the bladder 12.

An inner surface 22 of the bladder 12 is proximate the bodily extremity and the bladder is worn as a sock. In this case, the bladder is worn over the foot 15, ankle 17 and part of the calf 19. Protrusions in the form of ribbed formations 24 are provided on the inner surface or sole 23 of the bladder 12 for massaging the foot 15.

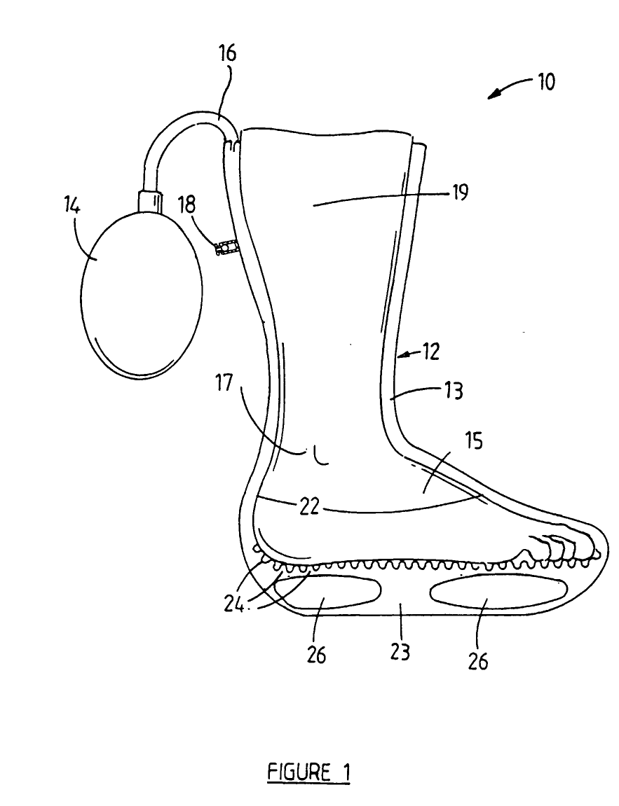

In a first embodiment of the invention (shown in FIG. 1), the valve 18 is a slow-release valve to enable the bladder 12 to deflate over time. A user can inflate the bladder 12 periodically by operating the air pump 14.

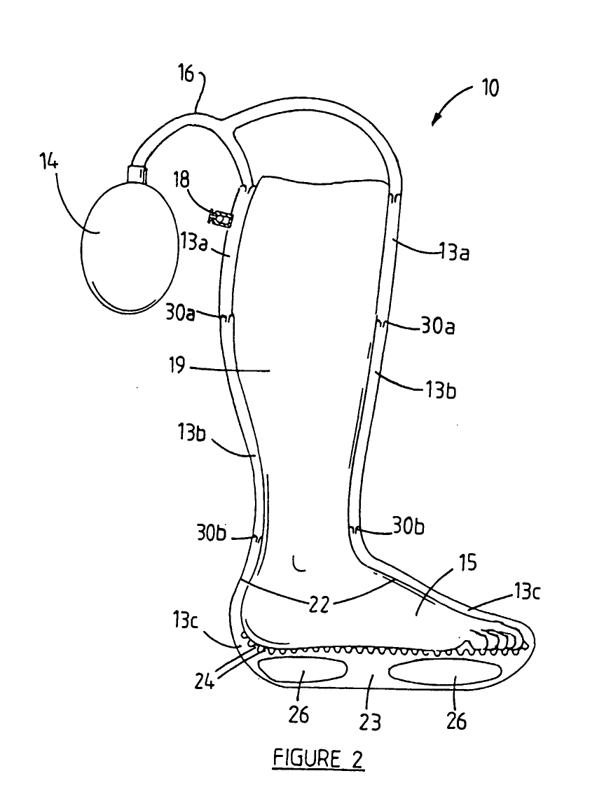

In a second embodiment of the invention (shown in FIG. 2), the inner cavity 13 of the bladder 12, is divided into a series of internal compartments 13a, 13b and 13c. The internal compartments 13a, 13b and 13c are in flow communication through pressure-release valves 30a and 30b.

The internal compartments 13a, 13b and 13c in conjunction with the pressure-release valves 30a and 30b are designed to maintain a decreasing pressure gradient from a distal portion to a proximal portion of the pressure sock 10. The compartment 13c located at the distal end of the pressure sock 10 is accordingly maintained at a relatively high pressure, typically 20 mmHg, with compartment 13b at a lower pressure, typically 14 mmHg and compartment 13a at a still lower pressure, typically 8 mmHg.

The pressure sock 10 of FIG. 1 and FIG. 2 further includes a set of pads 26, located in sole of the pressure sock 10 and are made of a resiliently deformable material, such as rubber.

The user exerts pressure on the pads 26 to stimulate airflow between the internal compartments 13a, 13b and 13c and through slow release valve 18 in the case of the second embodiment shown in FIG. 2, or to stimulate airflow through the slow-release valve 18 in the case of the first embodiment shown in FIG. 1.

In use, the pressure sock 10 of FIGS. 1 and 2 is fitted around the foot 15, ankle 17 and part of the calf 19. The air pump 14 is manually operated to inflate the bladder 12. The bladder 12 of the pressure sock illustrated in FIG. 1 is inflated to a comfortable pressure. The bladder 12 deflates slowly so that a decreasing pressure is applied to the foot 15, ankle 17 and calf 19 over a length of time. To expedite deflation, the user can exert pressure on the pads 26, which stimulates airflow from the internal cavity 13 of the bladder 12 and through the slow-release valve 18. The user is able to inflate the bladder 12 periodically or when necessary.

The compartment 13a of the pressure sock illustrated in FIG. 2 is inflated with the manual pump. When inflated to a predetermined pressure, typically 8 mmHg, the pressure-release valve 30a facilitates the flow of air into compartment 13b. Similarly, compartment 13c is inflated when pressure release valve 30b releases at approximately 14 mmHg.

It will be appreciated that the pressure sock shown in FIG. 1 does not have to include a slow release valve but may include a pressure release valve to ensure that the bladder could not be inflated to a high pressure.

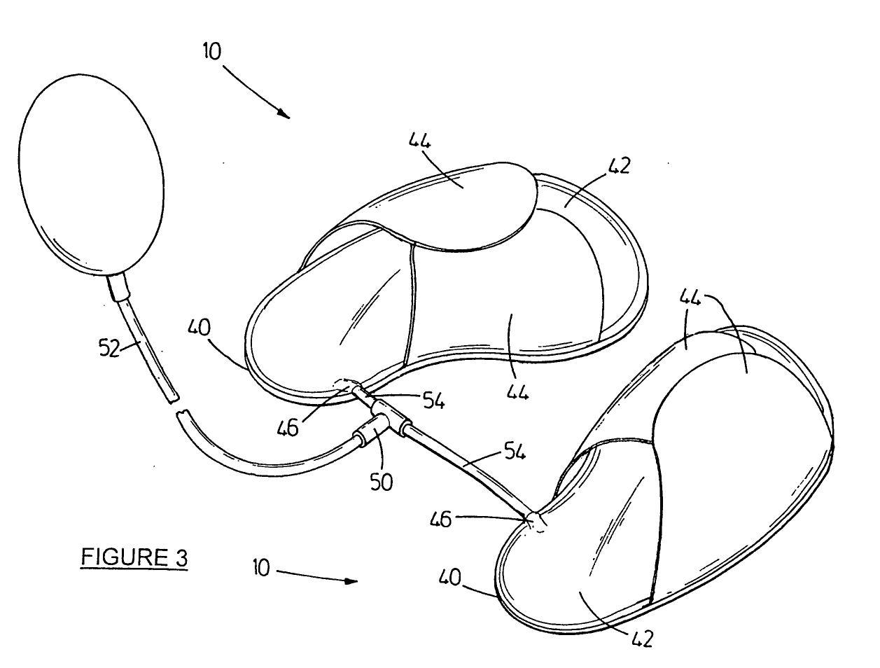

FIG. 3 shows a third embodiment of pressure socks. Each pressure sock includes a sole portion 40 locatable underneath a sole of the foot of a wearer, a bladder 42 and attachment means in the form of foot straps 44. The foot straps 44 are attached to sides of the sole portions 40. The bladders are made of plastics material and include filler openings 46. The bladders 42 substantially span the upper surface area of the sole portions 40.

The sole portions are made of a flexible, rigid or semi-rigid material. Alternatively, the walls of the bladder could be dimensioned to form an integral sole portion thereby obviating the need to have a separate or non-integral sole portion.

The foot straps 44 include hook and pile or Velcro® fasteners so that they are releasably attachable to the foot of a wearer. The foot straps 44 overlap to accommodate use of the hook and pile fasteners and can thus also be adjusted to fit different sizes.

The two bladders 42 of the two socks 10 shown in FIG. 3 are inflated with a manually operated hand pump being connected to a tube 52 terminating in a T-piece 50. Two tubes 54 extending from the T-piece are, in turn, sealingly connected in each filler opening 46. Inner cavities of each bladder are thus filled with air by operating the pump.

A slow release or pressure release valve may be included with each bladder so that an inner cavity of each bladder is in flow communication with the outside through such a valve.

It is envisaged that the pressure sock arrangement shown in FIG. 3 will be convenient to use with both feet. The bladders of the pressure socks of FIG. 3 will also maintain equal pressure there between as the bladders are in flow communication through the tubes 54 and the T-piece 50. The arrangement of FIG. 3 will further have the advantage that, when one foot is pushed down harder than the other, the other foot will experience a pressure increase due to such movement. This will result in a change of pressure in different feet at different times, due to different movement of the feet and will stimulate blood flow.

Pressure exerted on the feet by the bladders of the arrangements shown in FIG. 3 is different to the pressure exerted by the bladders of the arrangements shown in FIGS. 1 and 2. The bladders of the socks of FIGS. 1 and 2 extend around the feet and are inflated equally around the feet whereas the bladders of the socks of FIG. 3 only exert pressure from underneath the soles of the feet.

It is envisaged that the pressure socks described herein will be useful in stimulating blood circulation in passengers traveling long distances by aeroplane or other transport or people being seated or stationary for long periods and will help to prevent deep vein thrombosis.

It will be appreciated that the invention is not limited to the precise details as described hereinbefore. For example, the manually operable air pump can be a foot pump; the pads could be comprised of sponge or could be hollow; and the valve need not be a slow-release valve. The manually operable pump can be replaced with an electric or alternate power source pump. The electric or alternate power source pump can be set to automatically and periodically inflate and deflate the sock. This cycle may be set by a user.

1. A pressure sock arrangement comprising two inflatable bladders each attachable to a foot of the human body and a manually operable air pump in fluid flow communication with the bladders for inflating the bladders to apply pressure to the feet to which the bladders are attached, the bladders also being in fluid flow communication.

2. A pressure sock arrangement as claimed in claim 1 in which the bladders include attachment means for attaching the bladders to the feet.

3. A pressure sock arrangement as claimed in claim 2 in which the attachment means are straps having releasably securable fasteners.

4. A pressure sock arrangement as claimed in claim 3 in which the fasteners are complementary hook and pile fasteners.

5. A pressure sock arrangement as claimed in any one of the preceding claims in which the bladders are attached to an operatively upper surface of a sole portion of the pressure sock.

6. A pressure sock as claimed in claim 5 in which the sole portion is flexible.

7. A pressure sock arrangement as claimed in claim 5 in which the sole portion is rigid.

8. A pressure sock arrangement as claimed in any one of the preceding claims in which each inflatable bladder includes a pressure release valve.

9. A pressure sock arrangement as claimed in any one of the preceding claims in which the inflatable bladders include a slow release valve.

10. A pressure sock arrangement as claimed in claim 1 in which the bladders are dimensioned to fit over at least part of a foot of the human body.

11. A pressure sock arrangement as claimed in claim 10 in which each bladder has protrusions on at least part of its inner surface which contacts part of the foot to which it is attached when in use.

12. A pressure sock arrangement as claimed in any one of claims 10 or 11 in which at least one pad is located in a sole of each bladder.

13. A pressure sock arrangement as claimed in any one of claims 10 or 12 in which an inner cavity of each bladder is divided into a number of compartments.

14. A pressure sock arrangement as claimed in claim 13 in which the compartments are in flow communication through a number of different pressure pressure release valves so that each bladder can be inflated to maintain different pressures in different compartments.

15. A pressure sock arrangement as claimed in any one of claims 10 to 14 in which at least part of an inner cavity of each bladder is in flow communication with a pressure release valve to allow air in that part of the cavity to escape from that part of the cavity at a predetermined pressure.

16. A pressure sock arrangement as claimed in any one of claims 10 to 14 in which at least part of an inner cavity of each bladder is in flow communication with a slow release valve to allow air in that part of the cavity to escape from that part of the cavity.

17. A pressure sock arrangement as claimed in any one of claims 14 to 16 in which a distally disposed compartment is maintained at a higher pressure relative to its adjoining proximally disposed compartment.
